(19) United States
(12) Patent Application Publication
(10) Pub. No.: US 2025/0136926 A1
(43) Pub. Date: May 1, 2025

(54) DRUG DELIVERY CARRIER COMPLEX TARGETING CENTRAL NERVOUS SYSTEM, PREPARATION METHOD AND APPLICATION THEREOF

(71) Applicant: AFFILIATED HOSPITAL OF NANTONG UNIVERSITY, Nantong (CN)

(72) Inventors: Wei SHI, Nantong (CN); Qianqian LIU, Nantong (CN); Xingjia ZHU, Nantong (CN); Youlang ZHOU, Nantong (CN); Bo YOU, Nantong (CN)

(21) Appl. No.: 19/004,254

(22) Filed: Dec. 28, 2024

Related U.S. Application Data
(63) Continuation of application No. PCT/CN2023/127797, filed on Oct. 30, 2023.

(30) Foreign Application Priority Data
Jul. 20, 2023 (CN) 202310892804.6

(51) Int. Cl. C12N 5/00; A61K 47/69; C12N 5/079

(52) U.S. Cl. CPC C12N 5/0006; A61K 47/6901; C12N 5/0622

(57) ABSTRACT

Disclosed are a drug delivery carrier complex targeting a central nervous system and a preparation method and an application thereof, relating to the technical field of biomedicine. The preparation method includes following steps: carrying out azide modification on carrier cells to obtain azide-modified carrier cells; modifying sulfhydryl group and cholesterol to two ends of therapeutic miRNA respectively, and then performing ligation reaction to modified therapeutic miRNA and an acryl complex to form a miRNA complex; and co-incubating the azide-modified carrier cells with the miRNA complex, and forming the drug delivery carrier complex through click chemistry reaction.

### CROSS-REFERENCE TO RELATED APPLICATIONS

This application is a continuation of PCT/CN2023/127797, filed Oct. 30, 2023, and claims priority of Chinese Patent Application No. 202310892804.6, filed on Jul. 20, 2023, the entire contents of which are incorporated herein by reference.

### TECHNICAL FIELD

The disclosure relates to the technical field of biomedicine, and in particular to a drug delivery carrier complex targeting a central nervous system and a preparation method and an application thereof.

### BACKGROUND

Blood-brain barrier (BBB) is a barrier separating brain cells from plasma formed by brain capillary walls and glial cells, and also a barrier between the cerebrospinal fluid and the plasma formed by choroid plexus. Blood-brain barrier strictly controls specific nutrients (such as amino acids, glucose, nucleosides and fatty acids) from the vascular system into the extracellular fluid of the central nervous system, and restricts the passage of harmful exogenous molecules (such as neurotoxins) to maintain the steady state of the central nervous system. At the same time, the blood-brain barrier also prevents certain drugs and macromolecular therapeutic drugs such as biopharmaceuticals from entering the brain, and the low level of drug concentration may affect the therapeutic efficiency of drugs. Therefore, an effective non-invasive technology is urgently needed to deliver biopharmaceuticals to the central nervous system efficiently.

At present, the techniques of targeting the central nervous system may be roughly divided into the following categories: receptor-mediated transcytosis (RMT), the application of neurotrophic virus, delivery system of nanoparticles, exosomes-mediated delivery system, and other delivery systems.

For the development of RMT delivery technology in the central nervous system, the ideal receptor should only be expressed on blood-brain barrier cells, and has high transport capacity mediated by selective ligands, and these ligands will not interfere with the natural function of the receptor and will not induce lysozyme degradation of normal pathway. Although such an “ideal” receptor has not been found yet, there are ongoing clinical trials of drugs targeting transferrin receptor (TfR), insulin receptor and low density lipoprotein (LDL) receptor related protein 1 (LRP1). These results may confirm whether RMT-based technology may safely and effectively deliver treatment-related drug doses to the central nervous system, while minimizing peripheral and/or off-target toxicity.

Neurotropic virus, as a kind of virus with affinity to nervous tissue, has also been used as a trial carrier to enter the central nervous system, including mainly adeno-associated virus (AAV) and lentivirus, while others include herpes simplex virus (HSV) and virus-like particles (VLPs). At present, the application of these viral carriers is limited to gene therapy, and usually depends on invasive drug delivery methods that require additional monitoring of patients, such as intrathecal or intrarenal injection. A new viral carrier spanning blood-brain barrier, such as some AAV variants, is expected to be developed to reduce the dependence on invasive treatment in neurosurgery. The problems that should be considered in the use of neurotrophic virus as a carrier include the purity of the carrier, regulatory elements and transgenic sequences, the use of non-integrated or self-inactivating carriers, the nature of packaged DNA, the route of administration, the formula, and the infusion volume and rate. More research should be conducted to make a strong safety assessment and select the optimal viral carrier.

Nanoparticle systems are widely used in the exploration of carriers, such as liposomes, polymeric nanoparticles and solid lipid nanoparticles (SLNPs). Studies have shown that non-targeted liposomes are more passively absorbed, which may be due to the damage of the integrity of the blood-brain barrier, rather than the active blood-brain barrier transport mechanism. The application of targeted liposomes, such as glutathione (GSH)-polyethylene glycol liposomes, has been proved to be highly effective in brain targeting in some preclinical studies. Nanoparticle polymers represented by poly(lactic-co-glycolic acid) (PLGA) have also been applied in neurodegenerative diseases and brain tumors. Overall, although the research shows that drug delivery with nanoparticles as the carrier has certain clinical value in the treatment of central nervous system diseases, it has to be noted that the success of most cases is achieved in the context of brain cancer, precisely when the integrity of the blood-brain barrier is compromised. The inherent properties of nanoparticles (including particle size, surface charge, etc.) will also affect drug delivery. The internal stability of nanoparticles is also very important, and the carrier needs to remain intact until it reaches the brain to produce curative effect. Degradation is a problem faced by many nanoparticle carriers. In a word, at present, there is no effective way to target nanoparticles to the brain and improve the blood concentration in the brain, and further studies are required.

Exosomes are small vesicles of cell origin with a diameter of 40-100 nanometer (nm), which may be secreted by most types of cells. Exosomes may contain various substances, including protein and nucleic acids. In recent years, more and more studies have proved that exosomes have the ability to deliver drugs to the central nervous system as a carrier, but there are still some problems to be solved to make the exosomes suitable for the treatment of central nervous system diseases. The first is the acquisition of exosomes, and the exosome content depends on the cells from which the exosomes are produced. Exosomes from different cells may cause different effects, and identifying the appropriate source of exosomes may help avoid potential side effects. Secondly, if there is no brain-targeted modification, only a small part of injected exosomes may reach the brain smoothly. However, the mechanism of exosomes passing through BBB is still unclear. The main mechanism of interaction between exosomes and cells is membrane fusion, and then their contents are sent to cells. Another important issue is the loading efficiency of exosomes. The existing loading strategies of exosomes are all inefficient, which will limit the maximum applicable dose of the load and make exosomes reach the therapeutic inefficiency level. These problems must be solved if exosomes are to be used as the delivery tool of central nervous system therapeutic drugs.

Cell-mediated brain transfer is a relatively new method and may be regarded as a special application of cell therapy in a wider context. These cells may also be modified to produce peptides, protein or ribonucleic acid (RNA) molecules related to treatment, or to carry liposomes that encapsulate drugs, which may be used for autologous, patient-specific treatment, and may also be used as allogeneic general cell therapy infused into systemic circulation. Immune cells such as macrophages and monocytes are suitable candidates for brain delivery of neuroinflammatory diseases or neurodegenerative diseases with neuroinflammatory components, because these cells are recruited to cross the blood-brain barrier during brain inflammation. These immune cells rely on their own “infiltration” process to change their shape to cross the blood-brain barrier.

At present, the exploration of cell-mediated delivery carrier for central nervous system is still in the early stage, and there are many unresolved problems. For example, it is not clear whether the number of cells that may cross the blood-brain barrier is enough for effective treatment, or whether the cells reach the brain regions related to treatment. Because only a small number of carrier cells may enter the brain through peripheral vein administration, most cells remain in other organs. Therefore, the distribution of carrier cells in other major peripheral organs is also a major issue that must be considered.

### SUMMARY

An objective of the disclosure is to provide a drug delivery carrier complex targeting a central nervous system and a preparation method and an application thereof, so as to solve the problems existing in the prior art. The drug delivery carrier complex has good targeting to the central nervous system and may be used for preparing drugs for treating central nervous system diseases.

In order to achieve the above objectives, the disclosure provides a following scheme.

The disclosure provides a preparation method of a drug delivery carrier complex targeting a central nervous system, including following steps:
carrying out azide modification on carrier cells to obtain azide-modified carrier cells;
modifying sulfhydryl group and cholesterol to two ends of therapeutic micro ribonucleic acid (miRNA) respectively, and then performing ligation reaction to modified therapeutic miRNA and an acryl complex to form a miRNA complex; and
co-incubating the azide-modified carrier cells with the miRNA complex, and forming the drug delivery carrier complex through click chemistry reaction.

In an embodiment, the carrier cells include microglia cells or neutrophil (NE) cells.

In an embodiment, the carrier cells are microglia cells.

In an embodiment, the therapeutic miRNA is miR-138-5p mimic.

In an embodiment, the acryl complex is dibenzocyclooctyne-polyethylene glycol-acrylate.

In an embodiment, a method for carrying out the azide modification on the carrier cells includes co-culturing the carrier cells with azide-modified mannose.

The disclosure also provides a drug delivery carrier complex targeting a central nervous system prepared by the preparation method.

The disclosure also provides an application of the drug delivery carrier complex in improving targeting of the central nervous system in a pharmaceutical process.

The disclosure also provides a drug for treating central nervous system diseases, including the drug delivery carrier complex.

The disclosure discloses the following technical effects.

According to the disclosure, a new and original drug delivery carrier complex targeting a central nervous system is constructed through a bioorthogonal labeling strategy based on metabolic sugar engineering and bioorthogonal click chemistry. The drug delivery carrier complex may smoothly cross the blood-brain barrier and has good targeting to the central nervous system. The disclosure develops a new drug-carrying system for preparing drugs for treating central nervous system diseases, so that central nervous system diseases may be treated by peripheral vein administration, thus avoiding the infection risk caused by in-situ injection administration of lateral ventricle.

### BRIEF DESCRIPTION OF THE DRAWINGS

In order to explain the embodiments of the disclosure or the technical scheme in the prior art more clearly, the drawings needed in the embodiments will be briefly introduced below. Obviously, the drawings described below are only some embodiments of the disclosure, and other drawings may be obtained according to these drawings without creative work for ordinary people in the field.

FIG. 1 is a schematic diagram of constructing a carrier complex according to the disclosure.

FIG. 2A shows the quantitative statistical results of fluorescence values after attaching miR-138-5p mimics to cell surfaces and adding CES2 in vitro.

FIG. 2B shows the quantitative analysis results of two cell vectors carrying miR-138-5p mimics to the central nervous system of rats.

FIG. 2C is the result of quantitative analysis of uptake by neurons of two cellular vectors carrying miR-138-5p mimics observed by confocal microscopy.

FIG. 2D shows the statistical results of gray values of CES2 expression in the injury area of MCAO rats by Western blot analysis.

FIG. 2E shows the statistical results of gray values of miR-138-5p downstream target gene expression by Western blot analysis, where all data represent the average±SEM of three independent experiments, P value uses two-way Analysis of variance (ANOVA) or Student t tests, n.s. P≥0.05, *P<0.05, **P<0.01, ***P<0.001, ****P<0.0001.

### DETAILED DESCRIPTION OF THE EMBODIMENTS

A number of exemplary embodiments of the disclosure will now be described in detail, and this detailed description should not be considered as a limitation of the disclosure, but should be understood as a more detailed description of certain aspects, characteristics and embodiments of the disclosure.

It should be understood that the terminology described in the disclosure is only for describing specific embodiments and is not used to limit the disclosure. In addition, for the numerical range in the disclosure, it should be understood that each intermediate value between the upper limit and the lower limit of the range is also specifically disclosed. Intermediate values within any stated value or stated range, as well as each smaller range between any other stated value or intermediate values within the stated range are also included in the disclosure. The upper and lower limits of these smaller ranges may be independently included or excluded from the range.

Unless otherwise specified, all technical and scientific terms used herein have the same meaning as commonly understood by one of ordinary skill in the art to which this disclosure relates. Although the disclosure only describes the preferred methods and materials, any methods and materials similar or equivalent to those described herein may also be used in the practice or testing of the disclosure. All documents mentioned in this specification are incorporated by reference to disclose and describe methods and/or materials related to the documents. In case of conflict with any incorporated document, the contents of this specification shall prevail.

It is obvious to those skilled in the art that many improvements and changes may be made to the specific embodiments of the disclosure without departing from the scope or spirit of the disclosure. Other embodiments will be apparent to the skilled person from the specification of the disclosure. The specification and embodiments of that disclosure are exemplary only.

The terms “comprising”, “including”, “having” and “containing” used in this disclosure are all open terms, which means including but not limited to.

### Description of Terms

The miR-138-5p mimic referred to in the disclosure is a chemically synthesized miRNA mimic, which may simulate endogenous miRNA and up-regulate miRNA activity, and is constructed and modified by Suzhou Genepharma Co., Ltd.

The dibenzocyclooctyne-polyethylene glycol-acryl (DBCO-PEG-AC) complex referred to in the disclosure refers to dibenzocyclooctyne-polyethylene glycol-acrylate, which is purchased from Shanghai ToYongBio Company.

### Embodiment 1

### 1. Method

### 1.1 Construction of Carrier Complex

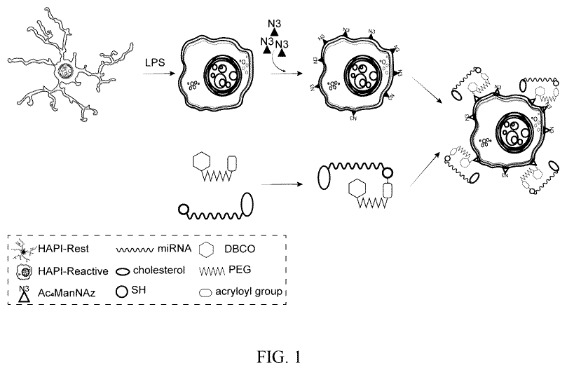

The construction strategy shown in FIG. 1 operates as follows.

### (1) Preparation and Pretreatment of Carrier Cells

Primary neutrophil cells (NE) are prepared from tibia and femur of a Sprague Dawley (SD) rat aged 8-12 weeks, and cultured in Roswell Park Memorial Institute (RPMI) 1640 medium (Gibco) containing 10% fetal bovine serum (FBS). Highly aggressively proliferating immortalized cells (HAPI cells) are purchased from Shanghai American Type Culture Collection (ATCC) cell bank. HAPI cells are cultured in Dulbecco's Modified Eagle Medium (DMEM) medium (Gibco) supplemented with 10% FBS and 100 unit per milliliter (U/mL) penicillin-streptomycin.

(2) Pre-Modification of Therapeutic miRNA

HAPI cells and NE cells cultured in step (1) are co-cultured with azide-modified mannose (CAS No.: 361154-30-5) for 1.5 hours (h), and co-incubation pretreatment is carried out before bioorthogonal labeling to form cell complexes, namely azide-modified HAPI cells and azide-modified NE cells respectively.

The two ends of miR-138-5p mimic are chemically modified in advance. The 5′ end of antisense strand of mimic is modified with sulfhydryl group, and 3′ end is modified with cholesterol. In order to facilitate fluorescence observation, 3′ end is then labeled with the fluorescent dye Cyanine 5 (CY5). The modified miR-138-5p mimic and dibenzocyclooctyne-polyethylene glycol-acryl (DBCO-PEG-AC) complex are incubated at 25 degrees Celsius (° C.) for 0.5 h, and the two simulated complexes are linked by thioether bond between sulfhydryl group and propylene group to form mimic complex.

### (3) Connection of the Carrier Cells and the Mimic Complex Through Click Chemistry Reaction

Azide-modified HAPI cells and azide-modified NE cells are incubated with the mimic complex at 25° C. respectively, and the HAPI cell carrier complex (mimic-HAPI) and NE cell carrier complex (mimic-NE) are formed by click chemistry reaction respectively.

1.2 the Carrier Complexes are Loaded with Therapeutic miRNA, and the Experimental Effect is Verified by In Vitro Fluorescence Observation, In Vivo Imaging, Fluorescence Observation and the Change of Downstream Protein Level.

### 2. Results

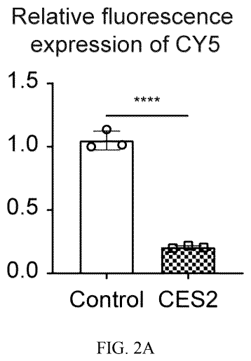

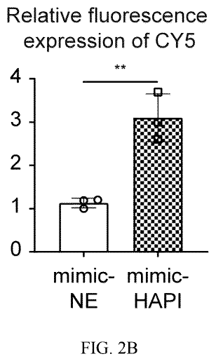

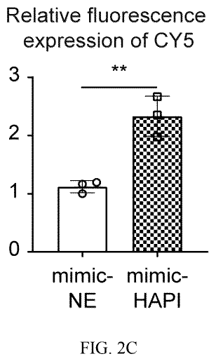

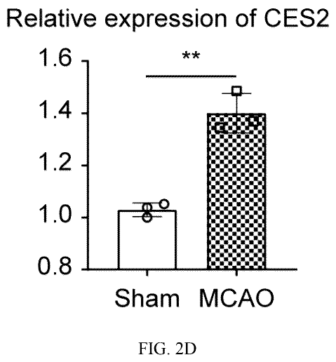

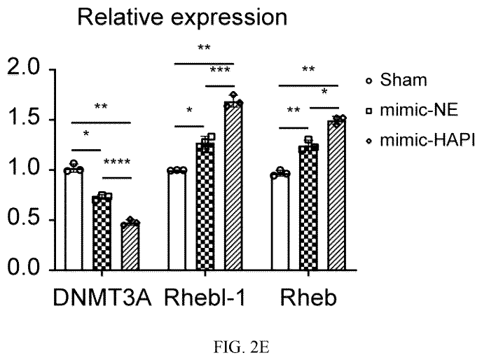

FIG. 2A-FIG. 2E show the results of in vitro and in vivo effect verification experiments of therapeutic miRNA carried by carrier complexes. After the construction of the carrier complexes is completed, it is observed through fluorescence microscope that miR-138-5p mimic is loaded onto the cell surfaces (FIG. 2A). Using the characteristic that inflammatory cells target the damaged inflammatory area, the disclosure selects different inflammatory cells (neutrophil cells NE and microglia cells HAPI) as carriers, and respectively discusses their efficiency of targeted delivery of miRNA to the damaged area. According to the disclosure, miR-138-5p mimic is loaded on the surfaces of NE cells or HAPI cells. The staining results also show that the miR-138-5p mimic labeled with CY5 may reach the ischemic damaged area (FIG. 2B) and be taken up by neurons (FIG. 2C). Therefore, the disclosure finds that both HAPI cells and NE cells may be used as carriers for targeting the central nervous system, and experiments prove that HAPI cells have better targeting, so the disclosure uses HAPI cells as carriers in subsequent experiments.

When miRNA is transported to the damaged area by HAPI cells, miRNA must be separated from the loaded cells to play its role. Therefore, the miRNA is modified by sulfhydryl group and connected with DBCO-PEG-AC by thioether bond. Because the cells in the damaged area break down and release esterases, such as carboxylesterases, these esterases may break the ester bonds in DBCO-PEG-AC, thereby separating the miRNA from the cell surface. In the past decades, the molecular characteristics of carboxylesterase (including CES1 and CES2) and its biological functions in vivo and exogenous metabolism have been widely studied. Recent studies show that CES is involved in the hydrolysis of a large number of endogenous esters. In vitro experiments with CES2 cells co-cultured with mimic-HAPI for 8 h show that mimic is separated from HAPI (FIG. 2A). In vivo experiments confirm that the expression of CES2 increases significantly in the ischemic injury area of MCAO rats (FIG. 2D), indicating that there is an internal environment of ester bond dissociation in vivo.

Finally, the expressions of deoxyribonucleic acid methyltransferase 3A (DNMT3A), Ras homolog enriched in brain (Rheb) and Ras homolog enriched in brain like 1 (Rhebl1) in the injury area of the treated MCAO rat is detected. The results show that miR-138-5p mimic may play a similar role after reaching the damaged area, reducing the expression of DNMT3A, thus increasing the level of Rhebl1. Similar to the previous results, HAPI cells have stronger targeting ability than NE cells (FIG. 2E).

The above-mentioned embodiments only describe the preferred mode of the disclosure, and do not limit the scope of the disclosure. Under the premise of not departing from the design spirit of the disclosure, various modifications and improvements made by ordinary technicians in the field to the technical scheme of the disclosure shall fall within the protection scope determined by the claims of the disclosure.

What is claimed is:

1. A preparation method of a drug delivery carrier complex targeting a central nervous system, comprising following steps:
carrying out azide modification on carrier cells to obtain azide-modified carrier cells;
modifying sulfhydryl group and cholesterol to two ends of therapeutic miRNA respectively, and then performing ligation reaction to modified therapeutic miRNA and an acryl complex to form a miRNA complex; and
co-incubating the azide-modified carrier cells with the miRNA complex, and forming the drug delivery carrier complex through click chemistry reaction.

2. The preparation method according to claim 1, wherein the carrier cells comprise microglia cells or neutrophil cells.

3. The preparation method according to claim 2, wherein the carrier cells are microglia cells.

4. The preparation method according to claim 1, wherein the therapeutic miRNA is miR-138-5p mimic.

5. The preparation method according to claim 1, wherein the acryl complex is dibenzocyclooctyne-polyethylene glycol-acrylate.

6. The preparation method according to claim 1, wherein a method for carrying out the azide modification on the carrier cells comprises co-culturing the carrier cells with azide-modified mannose.

7. A drug delivery carrier complex targeting a central nervous system prepared by the preparation method according to claim 1.

8. A drug for treating central nervous system diseases, comprising the drug delivery carrier complex according to claim 7.
